(19) United States
(12) Patent Application Publication
(10) Pub. No.: US 2023/0157784 A1
(43) Pub. Date: May 25, 2023

(54) SURGICAL MARKING GUIDE AND METHODS OF USE

(71) Applicant: The Board of Supervisors of Louisiana State University and Agricultural and Mechanical College, Baton Rouge, LA (US)

(72) Inventor: Gerhard S. Mundinger, New Orleans, LA (US)

(21) Appl. No.: 17/910,734

(22) PCT Filed: Mar. 11, 2021
(86) PCT No.: PCT/US2021/021946

§ 371 Date: Sep. 9, 2022

Related U.S. Application Data
(60) Provisional application No. 62/988,205, filed on Mar. 11, 2020.

(51) Int. Cl. A61B 90/00

(52) U.S. Cl. CPC A61B 90/39; A61B 2090/3908; A61B 2090/395

(57) ABSTRACT

The present invention is directed to a surgical marking guide. The surgical marking guide can be configured for use in the harvest of a nipple areolar complex graft, the implant of a nipple areolar complex graft, or both the harvest and implant of a nipple areolar complex. Methods of use of the surgical marking guide are also disclosed herein.

### CROSS-REFERENCE TO RELATED APPLICATIONS

This application claims priority from U.S. Provisional Application No. 62/988,205 filed on Mar. 11, 2020, the entire contents of which are incorporated herein by reference.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Female to male transgender chest surgery is an increasingly common procedure in which the natal female breasts and nipples are altered to a more masculine appearance. Most commonly, mastectomies are performed, and the nipple areolar complex (NAC) is removed, reduced in size and shape, and repositioned on the chest wall in a more masculine location. The ideal male nipple is generally circular in shape and has a size of about that of a nickel. However, when a circular NAC graft is harvested and the NAC graft is sewn to the chest wall as a circle, the skin tension on the chest wall pulls the nipple into an oval shape with the long axis of the oval oriented vertically. This results in an unnatural nipple appearance, regardless of whether the patient is a man or woman, cis or trans.

### FIELD OF THE INVENTION

The present invention relates to a surgical marking guide and methods of use, thereof. In embodiments, the surgical marking guide is configured for use in the harvest and implant of a nipple areolar complex graft.

### SUMMARY OF THE INVENTION

In one aspect, the present invention provides a surgical marking guide that comprises a first marking surface, a second marking surface, and a body. In embodiments, the body is integral with the first and second marking surfaces and extends therebetween. A through-opening can extend longitudinally from the first marking surface, through the body, and through the second marking surface.

In embodiments, the first marking surface is configured to mark an area for harvest of a nipple areolar complex graft. The second marking surface can be configured to mark an area for implant of a nipple areolar complex graft.

The first marking surface can comprise a substantially circular diameter. In certain embodiments, the first marking surface comprises a diameter that is about the same as that of an average adult male human nipple areolar complex. The diameter of the first marking surface can be between about 1 cm and about 4 cm. The first marking surface can comprise a diameter of about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, or about 2.9 cm.

In certain embodiments, the second marking surface comprises a substantially oval diameter. The second marking surface can comprise a horizontal diameter and a vertical diameter. In embodiments, the horizontal diameter is larger than the vertical diameter. The horizontal diameter can be up to 140% of the diameter of an average adult male human nipple areolar complex. The vertical diameter can be at least 60% of the diameter of the average adult male human nipple areolar complex. In one embodiment, the horizontal diameter is about 114% of the diameter of the average adult male human nipple areolar complex. The vertical diameter can be about 68% of the diameter of the average adult male human nipple areolar complex. In embodiments, the horizontal diameter is up to 4 cm. The vertical diameter can be as small as 0.5 cm. In certain embodiments, the horizontal diameter is about 2.0 cm, about 2.1 cm, about 2.2 cm, about 2.3 cm, about 2.4 cm, about 2.5 cm, about 2.6 cm, about 2.7 cm, about 2.8 cm, or about 2.9 cm. The vertical diameter can be about 1.0 cm, about 1.2 cm, about 1.3 cm, about 1.4 cm, about 1.5 cm, about 1.6 cm, about 1.7 cm, about 1.8 cm, or about 1.9 cm. In one embodiment, the horizontal diameter is about 2.5 cm and the vertical diameter is about 1.5 cm.

In various embodiments, the first marking surface and the second marking surface each comprise a means for marking the skin of a patient. The means for marking the skin of the patient can include a ridged surface, an inked surface, or a combination thereof.

The guide can comprise any material suitable for use on sterilized fields or suitable for use during surgical operation. In embodiments, the guide comprises a sterilizable material. The guide can comprise a surgical metal, a medical-grade polymer, or a combination thereof. In embodiments, the surgical metal comprises stainless steel, titanium, tantalum, gold, platinum, palladium, or a combination thereof.

The surgical marking guide can be reusable. In alternative embodiments, the guide is disposable.

Disclosed in another aspect is a method of grafting a nipple areolar complex to the chest of a patient in need thereof. In embodiments, the method comprises marking an area for a nipple areola complex graft using a first marking surface of a surgical marking guide in accordance with any of the embodiments described in this disclosure. The method can also include the step of harvesting a nipple areolar complex graft. In embodiments, the method comprises marking an area for implant of the nipple areolar complex graft on a patient. In certain embodiments, marking the area for implant is performed using a second marking surface of a surgical marking guide in accordance with any of the embodiments described in this disclosure. The method can further include the step of implanting the nipple areolar complex on the chest of the patient within the marked area for implant.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 provides a schematic top perspective view of a marking guide in accordance with an embodiment of the present invention.

FIG. 2 shows a schematic side view of the FIG. 1 marking guide. In this view, an elliptical second marking surface is seen down its minor axis such that the body of the second marking surface appears wider than the first marking surface.

FIG. 3 shows an alternate schematic side view of the FIG. 1 marking guide. In this view, the marking guide is rotated 90° as compared to the FIG. 2. The elliptical second marking surface is seen down its major axis such that the body of the second marking surface appears narrower than the first marking surface.

FIG. 4 provides a schematic top view of the FIG. 1 marking guide. In this view, the first marking surface is visible, and a portion of the body that correlates with the major axis of the second marking surface can be seen extending horizontally in the background.

FIG. 5A provides a schematic top perspective view of a marking guide in accordance with one embodiment of the present invention.

FIG. 5B shows a schematic bottom perspective view of the FIG. 5A embodiment.

FIG. 6A provides a top view of the presently disclosed marking guide under yet another embodiment. This view shows four orienting extensions protruding outwardly from the outside edge of the bottom surface.

FIG. 6B provides a bottom view of the embodiment shown in FIG. 6A. Four orienting extensions can be seen along the lateral edge of the second marking surface and extending outwardly therefrom.

FIG. 6C shows a schematic side view of the FIG. 6A marking guide. In this view, three of the four orienting extensions shown in FIG. 6B can be seen extending outwardly from the bottom surface of the guide.

FIG. 6D shows a top perspective view of the marking guide under the FIG. 6A embodiment.

FIG. 7A shows a schematic top view of a marking guide in accordance with another embodiment. Two reticle bars can be seen, one extending across the major axis and the other extending across the minor axis of the body of the marking guide. Two orienting extensions are also shown extending from the major axis of the marking guide.

FIG. 7B shows a schematic side view of the FIG. 7A marking guide. In this view, an elliptical second marking surface is seen down its minor axis. Two orienting extensions are shown extending outwardly from the major axis of the marking guide, and a third orienting extension can be seen extending toward the viewer from the minor axis of the marking guide.

FIG. 8 provides a schematic overview of female to male transgender chest surgery under one embodiment.

FIG. 9A provides a top view of a surgical marking guide under one embodiment being used to mark an area for implantation of a nipple areolar complex graft material.

FIG. 9B provides a view of a surgical filed immediately following use of a surgical marking guide under one embodiment for bilateral resection and implant of a nipple areolar complex graft material during a female to male transgender chest surgery.

FIG. 10A shows a close-up view of a patient immediately following use of a surgical marking guide under one embodiment for resection and implant of a nipple areolar complex graft material during a female to male transgender chest surgery.

FIG. 10B shows a close-up view if the patient in FIG. 10A three months following the use of the presently disclosed surgical marking guide during a female to male transgender chest surgery.

FIG. 11A provides a view of a patient's chest prior to a female to male transgender surgery.

FIG. 11B shows a view of the FIG. 11A patient's chest three months following the use of one embodiment of the presently disclosed surgical marking guide to perform a female to male transgender chest surgery.

FIG. 12A provides a view of a patient's chest prior to a female to male transgender surgery under another embodiment.

FIG. 12B shows a view of the FIG. 12A patient's chest three months following the use of a surgical marking guide under one embodiment to perform a female to male transgender chest surgery.

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” can refer to the term approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient” as used herein can include all members of the animal kingdom including, but not limited to, mammals, animals (e.g., cats, dogs, horses, swine, etc.) and humans.

The term “tissue” as used herein can refer to any conglomeration of cells along with the extracellular matrix that work in concert to carry out a specific function. In embodiments, tissue includes nervous tissue, epithelial tissue, connective tissue, muscular tissue, or a combination thereof. Tissue can include dermis, epidermis, subcutaneous fat, fascia, or any combination thereof. In certain embodiments, the tissue may be injured or diseased. Injured or diseased tissue can refer to any tissue that is inflamed, dry, cancerous, wounded, abraded, eroded, burned, fractionated, or has been subjected to any other type of tissue injury or disease or combinations thereof. Injured or diseased tissue can refer to any of various skin conditions known in the art.

As used herein, the term “medical procedure” can refer to any course of action that is intended to achieve a desired result in the field of healthcare. In certain embodiments, medical procedures can include cancer treatment, cosmetic procedures, lesion treatment, burn care, or a combination thereof. Lesions can include cancerous or precancerous lesions. Non-limiting examples of pre-cancerous lesions include actinic keratosis, actinic cheilitis. Examples of cancerous lesions include but are not limited to melanoma, basal cell carcinoma, or squamous cell carcinoma. The term “medical procedure” can further encompass any procedure used in a research setting to study a health-related condition or to study or develop treatments for a health-related condition.

As used herein, the term “cosmetic procedure” can refer to any procedure intended to alter the physical appearance of a subject or a subject's tissue. In embodiments, a cosmetic procedure includes any aesthetic procedure. Cosmetic procedures can be non-invasive or minimally invasive procedures. Cosmetic procedures can include any procedure known by one of skill in art to be a cosmetic or aesthetic procedure. Exemplary cosmetic procedures include but are not limited to treatment of skin conditions, treatment of skin damage, cosmetic reconstruction, or a combination thereof. In embodiments, cosmetic procedures include chest wall reconstruction.

As used herein, the term major axis can refer to the widest span of an oval or ellipse.

As used herein, the term minor axis can refer to the narrowest span of an oval or ellipse.

Female to male transgender chest surgery is an increasingly common procedure in which the natal female breasts and nipples are altered to a more masculine appearance. Most commonly, mastectomies are performed, and the nipple areolar complex (NAC) is removed, reduced in size and shape, and repositioned on the chest wall in a more masculine location. The size of the male areola approximates a circle with about a 2.2 cm diameter. However, because of the underlying pectoralis muscle pull, the NAC should be sewn in as an oval with a horizontal diameter of about 2.5 cm and a vertical diameter of about 1.5 cm (Agarwal et al). Over time, the pull of the underlying pectoralis major muscle and tension across the closure rounds the oval out to a circle of 2.2 cm diameter, approximating the “ideal” cisgender male nipple.

No marking guide for either the a.) areola harvest or b.) areola inset exists in transgender female-to-male chest surgery or cisgender gynecomastia procedures.

In various exemplary embodiments, the presently disclosed surgical marking guide is configured to mark an area on a patient for resection of a graft material. The marking guide can be further configured to mark an area on a patient for implant of a graft material. The guide is configured to permit accurate placement by maintaining visualization of the underlying skin while in use. The presently disclosed surgical marking guide provides accurate and sterile resection and implant of graft material. In embodiments, the surgical marking guide is configured to mark a nipple areolar complex for resection as graft material, mark an area for implant of a nipple areolar complex graft material, or a combination thereof.

The presently disclosed surgical marking guide allows for replicable resizing of feminine-appearing nipples to a masculine size and shape, accurate marking of both the NAC harvest and inset, and can significantly reduce operative time.

Studies have shown that the ideal male nipple is a perfect circle and is about the size of a nickel. However, when a nipple is harvested for grafting as a circle, and sewn to the chest wall as a circle, the skin tension on the chest wall pulls the nipple into an oval with the long axis of the oval oriented vertically. This results in an unnatural nipple appearance, regardless of whether the patient is a man or woman, cis or trans.

Implanting the circular nipple graft as an oval, can overcome this problem. In various embodiments, utilizing the skin's natural tension across the incision, and with the right orientation of the oval, the body's natural healing processes and contracture of the graft permits the implanted nipple areolar graft complex to obtain a more circular appearance with eccentricity in a horizontal orientation. This produces a nipple that appears more masculine, leading to higher patient satisfaction and superior aesthetic results. In various embodiments, the presently disclosed surgical marking guide and methods of use allow for marking an ideally sized circular nipple graft, and facilitates marking of the oval for insetting the nipple graft on the chest wall in the proper orientation to achieve these results.

### Description of Selected Embodiments

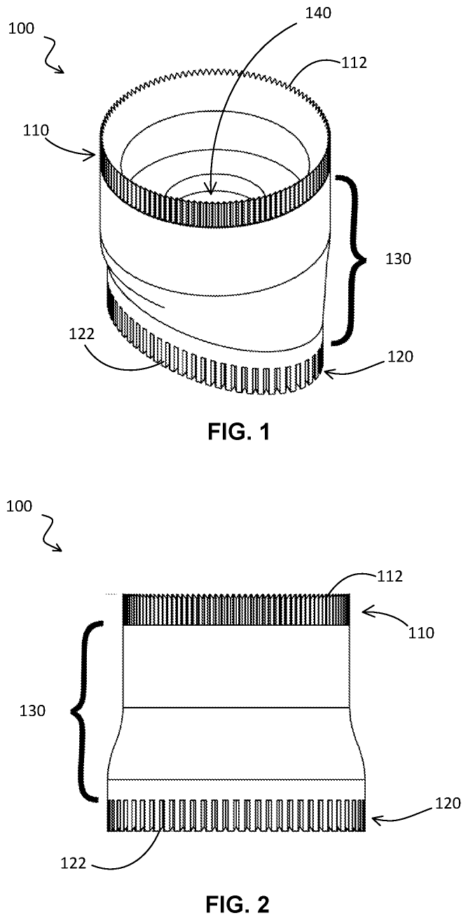

FIG. 1 provides a schematic top perspective view of a surgical marking guide 100 in accordance with an embodiment of the present invention. In the FIG. 1 embodiment, the marking guide 100 comprises a first marking surface 110 and a second marking surface 120 with a body 130 extending between the first and second marking surfaces 110, 120.

The exemplary embodiment of FIG. 1 shows a hollow marking guide 100 with an opening 140 that extends from the first marking surface 110, through the body 130, and continues through the second marking surface 120. In such embodiments, the opening serves as a window 140 to provide the practitioner with an unobstructed or minimally obstructed view of the patient's skin. This window 140 promotes accurate placement of the surgical marking guide 100, by permitting the practitioner to clearly see the skin of the patient while marking the surgical area. Such a design is particularly useful in cosmetic procedures wherein minor misalignments can produce unsatisfactory results.

FIG. 2 shows a schematic side view of the FIG. 1 marking guide 100. In this view, an elliptical second marking surface 120 is seen down its minor axis such that the portion of the body 130 near the second marking surface 120 appears wider than the portion of the body 130 near the first marking surface 110.

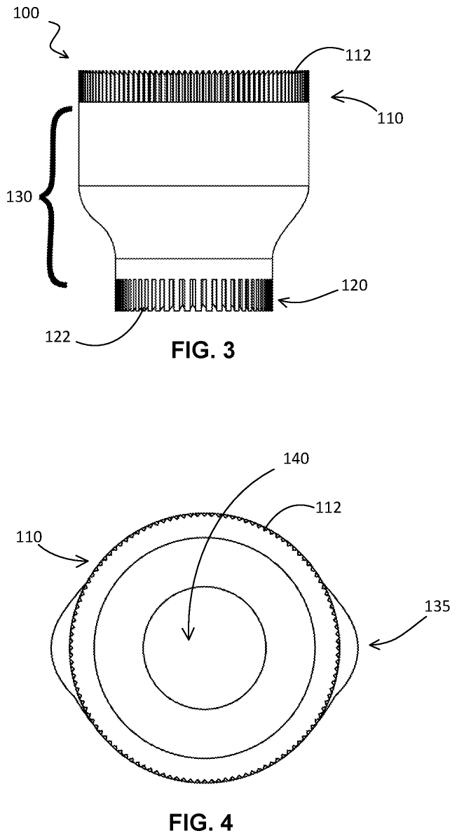

FIG. 3 shows an alternate schematic side view of the FIG. 1 marking guide 100, wherein the marking guide 100 is rotated 90° as compared to the FIG. 2 configuration. The elliptical second marking surface 120 is seen down its major axis such that the portion of the body 130 near the second marking surface 120 appears narrower than the portion of the body 130 of the first marking surface 110.

FIG. 4 provides a schematic top view of the FIG. 1 marking guide 100. This view is taken from directly above the marking guide 100 to reveal the substantially circular shape of the first marking surface 110. The pointed tips of the ridges disposed upon the ridged surface 112 are seen, and the full length of the viewing window 140 can be seen extending downward though the marking guide 100. A portion of the body that correlates with the major axis 135 of the second marking surface can be seen extending horizontally in the background to reveal the general elliptical shape of the second marking surface in the present embodiment.

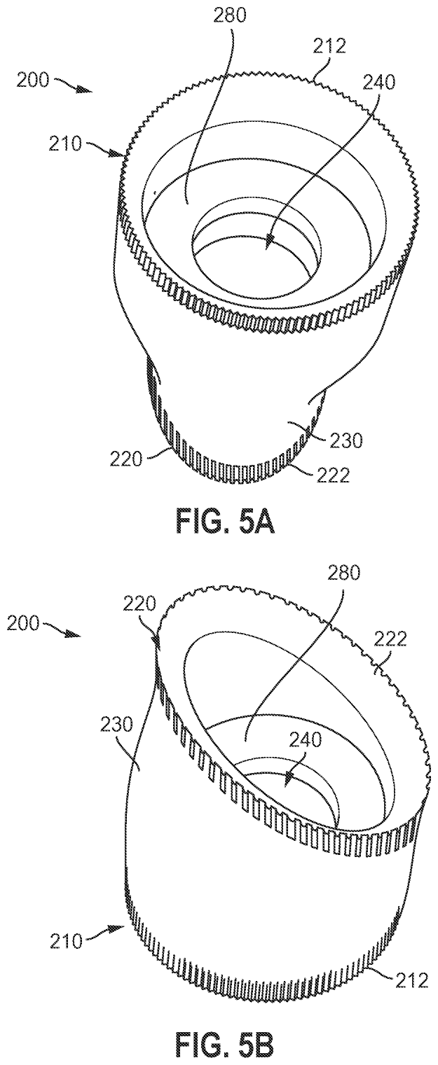

FIG. 5A provides a schematic top perspective view of a surgical marking guide 200 in accordance with another embodiment of the present invention. In the FIG. 5A embodiment, the marking guide 200 comprises a first marking surface 210 and a second marking surface 220 (more clearly seen in FIG. 5B) with a body 230 extending between the first and second marking surfaces 210, 220. As shown the first marking surface 210 can comprise a generally circular shape.

FIG. 5B shows a bottom perspective view of the FIG. 5A surgical marking guide 200. In this view, the second marking surface 220 is more clearly seen. As shown, the second marking surface 220 can comprise an oval shape.

In the FIG. 5 embodiment, the bottom surface 220 and the top surface 120 both comprise a means for marking the skin of a patient 222, 212 which is discussed in more detail below. The exemplary embodiment of FIG. 5 shows a hollow marking guide 200 with an opening 240 that extends from the first marking surface 210, through the body 230, and continues through the second marking surface 220. As discussed above, this opening serves as a window 240 to provide the practitioner with an unobstructed or minimally obstructed view of the patient's skin. As shown in this embodiment, the interior of the body 230 of the marking guide can comprise a support structure 280 that at least partially resides therein. In such embodiments, the window 240 extends through this support structure 280. In embodiments, the support structure 280 is integral with the body 230 of the marking guide.

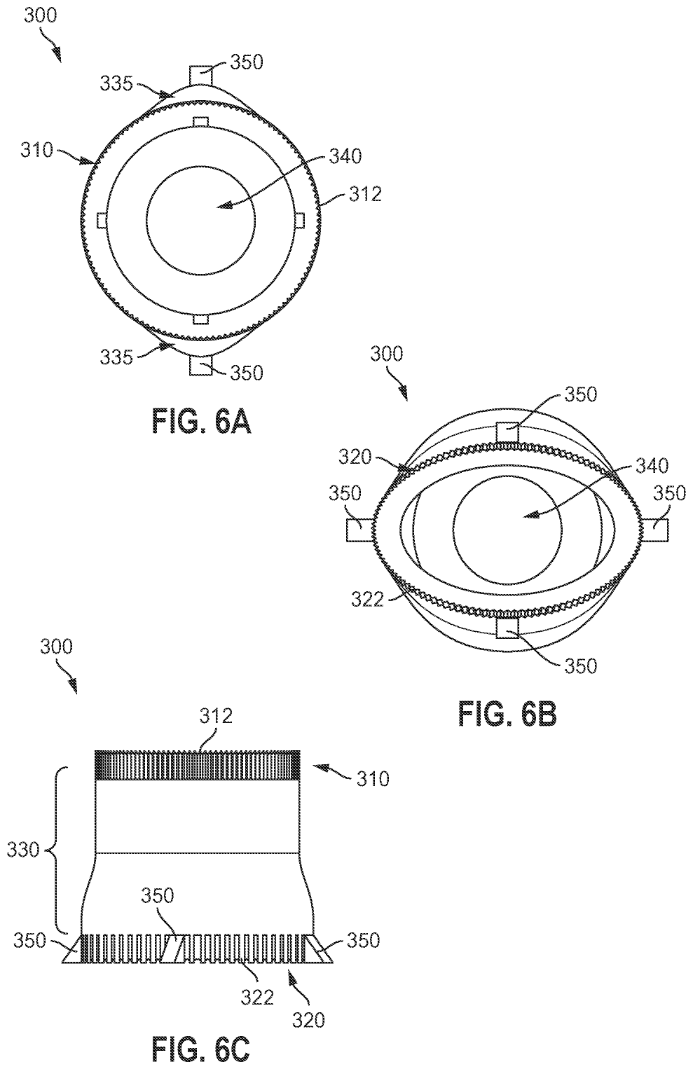

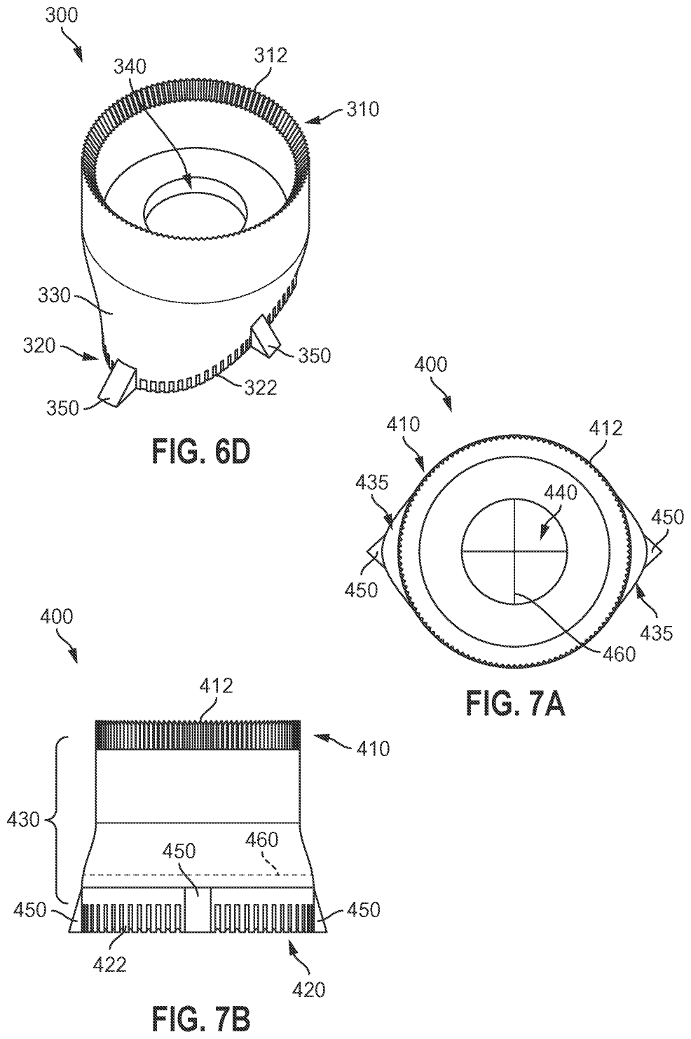

FIGS. 6A-6D show the marking guide in another embodiment 300 of the present invention. This embodiment comprises a first marking surface 310, a second marking surface 320, and a body 330 extending therebetween. In addition, the surgical marking guide 300 of the FIG. 6 embodiment comprises an opening that extends vertically through the guide 300, and serves as a window 340 for an unobstructed view of the surgical field. As shown in the FIG. 6 embodiment, the surgical marking guide 600 can include one or more orienting extensions 350. In embodiments, these orienting extensions 350 are configured to assist the practitioner in placement of the surgical marking guide during implantation of a graft material. The orienting extensions 350 fins that extend laterally from the second marking surface 320 of the surgical marking guide.

FIGS. 7A and 7B provide a schematic view of a surgical marking guide under another embodiment 400. The view of FIG. 7A is taken from directly above the marking guide 400 to reveal the substantially circular shape of the first marking surface 410. The pointed tips of the ridges disposed upon the ridged surface 412 are seen, and the full length of the viewing window 440 can be seen extending downward though the marking guide 400. A portion of the body that correlates with the major axis 435 of the second marking surface (more clearly seen in FIG. 7B) can be seen extending horizontally in the background to reveal the general elliptical shape of the second marking surface 420. Orienting extensions 450 can be seen extending laterally from a portion of the body that correlates with the apex of the major axis 435 of the second marking surface. In embodiments, additional orienting extensions are placed on the portion of the body that correlates with apex of the minor axis of the second marking surface (seen at 350 in FIG. 6B).

In embodiments, the orienting extensions 350, 450 extend laterally up to about 5 mm from the outside surface of the surgical marking guide 300, 400. The orienting extension 350, 450 can extend less than 1 mm from the outside surface of the surgical marking guide 300, 400. In various embodiments, the orienting extensions 350 extend up between about 1 mm and about 5 mm from the outside surface of the surgical marking guide 350. In embodiments, the orienting extensions 350 are about 1 mm long, about 2 mm long, about 3 mm long, about 4 mm long, or about 5 mm long. In the FIG. 6 and FIG. 7 embodiments, the orienting extensions 350, 450 are disposed on the apexes of the major axis and the minor axis of the second marking surface 320, 420; however, the orienting extensions 350, 450 can be placed at any point along the second marking surface 320, 420.

The FIG. 7A embodiment also includes a reticle 460, which can serve to orient the surgical marking guide 400 during use. The reticle 460 can extend across the interior space 440 of the marking guide 400. In embodiments, the reticle 460 is disposed adjacent to the second marking surface (more clearly seen in FIG. 7B at 420) and within the interior space 440. In alternate embodiments, the reticle 460 is disposed closer to the first marking surface. In certain embodiments, the reticle 460 is located the interior space 440 at about halfway between the first marking surface 410 and the second marking surface 420—in about the middle of the body 430.

In embodiments, the reticle 460 comprises at least one arm that extends from one side of the interior wall of the surgical marking guide 400, across the interior space 440, and terminates on the opposite side of the interior wall of the surgical marking guide 400. In embodiments, the reticle 460 comprises two arms. As shown in the embodiment of FIG. 7, the reticle 460 can comprise at least two arms that each traverse the interior space 440 of the surgical marking guide 400. As shown, the arms of the reticle 460 can be oriented such that they are substantially perpendicular to one another. In such embodiments, the arms of the reticle 460 form a plus-shaped guide across the viewing window 440, which can assist with accurate placement of the nipple graft. At least one arm of the reticle 460 can comprise a length that is sufficient to traverse the interior space 400 of the surgical marking guide 400. In embodiments, at least one arm of the reticle 460 is up to about 5 mm in length. At least one arm of the reticle 460 can comprise a length of less than about 1 mm. In various embodiments, at least one arm of the reticle 460 comprises a length between about 1 mm and about 5 mm. In embodiments, at least one arm of the reticle 460 is about 1 mm long, about 2 mm long, about 3 mm long, about 4 mm long, or about 5 mm long.

As shown in the embodiments of FIGS. 1-7, the first and second marking surfaces 110, 210, 310, 410, 120, 220, 320, 420 can be equipped with a means for marking the skin of a patient 112, 212, 312, 412, 122, 222, 322, 422. In embodiments, the marking means comprises a ridged surface, an inked surface, or a combination thereof. As shown in the embodiments of FIGS. 1-7, the marking means can comprise a ridged surface 112, 212, 312, 412, 122, 222, 322, 422 comprising a plurality of substantially parallel grooves and ridges. In embodiments, at least one of the ridges can comprise a sharpened or pointed tip. In operation, the ridged surface is depressed against the skin of a patient such that the pointed tips create a plurality of indentations thereon to mark a surgical area. In embodiments, the marked surgical area can comprise (1) tissue from the patient to be resected as a graft material, (2) an implantation site for inset of a graft material, or (3) a combination thereof. As shown in the FIGS. 1-7 embodiments, the first marking surface 110, 210, 310, 410 can comprise a first plurality of ridges 112, 212, 312, 412 configured to mark a surgical area to be resected as a graft material and the second marking surface 120, 220, 320, 420 can comprise a second plurality of ridges 122, 222, 322, 422 configured to mark an implantation site for inset of the resected graft material.

In embodiments, the first marking surface 110, 210, 310, 410 can comprise a shape that is distinct form that of the second marking surface 120, 220, 320, 420. In the pictured embodiments, the first marking surface 110, 210, 310, 410 is substantially circular in shape, and the second marking surface 120, 220, 320, 420 comprises a substantially elliptical or oval shape.

In embodiments, the surgical marking guide is configured for use in the transplant of a nipple areolar complex. The surgical marking guide can be configured for the harvest and inset of a nipple areolar complex in a manner that promotes a masculine appearance. By way of example, the guide can be used in female to male transgender surgeries, cisgender gynecomastia procedures, or a combination thereof. In certain embodiments, the surgical marking guide is configured for use in the surgical construction of congenitally or traumatically absent nipples.

In embodiments, the at least one marking surface is configured to mark an area for harvest of a nipple areolar complex graft, at least one marking surface is configured to mark an area for implant of a nipple areolar complex graft, or a combination thereof. In one embodiment, the first marking surface is configured to mark an area for harvest of a nipple areolar complex graft.

The first marking surface can comprise a diameter that is about the same as that of an average adult male human nipple areolar complex. The first marking surface can comprise a diameter that ranges from about 50% to about 150% the size of an average adult male human nipple areolar complex. The first marking surface can comprise a diameter that is about 70%, about 80%, about 90%, about 100%, about 110%, about 120%, or about 130% of the diameter of the average adult male nipple areolar complex. In embodiments, the first marking surface comprises a diameter between about 1 cm and about 3 cm. The first marking surface can comprise a diameter of about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, or about 2.9 cm. In one embodiment, the first marking surface comprises a diameter of about 2.2 cm.

The second marking surface can be configured to mark an area for implant of a nipple areolar complex graft. The second marking surface can comprise a substantially oval diameter. In one embodiment, the second the second marking surface comprises a horizontal diameter and a vertical diameter and the horizontal diameter is larger than the vertical diameter. In an alternate embodiment, the horizontal diameter is shorter than the vertical diameter. The second marking surface can comprise a major axis that corresponds with the horizontal diameter of the of the marking surface. a minor axis that corresponds with the vertical diameter of the marking surface, or vice versa.

The horizontal diameter of the second marking surface can be up to 140% of the diameter of an average adult male human nipple areolar complex. In embodiments, the vertical diameter of the second marking surface is at least 60% of the diameter of the average adult male human nipple areolar complex. In one embodiment, the horizontal diameter of the second marking surface is about 114% of the diameter of the average adult male human nipple areolar complex and the vertical diameter is about 68% of the diameter of the average adult male human nipple areolar complex.

In embodiments, the horizontal diameter of the second marking surface is up to 4 cm. The horizontal diameter can be about 2.0 cm, about 2.1 cm, about 2.2 cm, about 2.3 cm, about 2.4 cm, about 2.5 cm, about 2.6 cm, about 2.7 cm, about 2.8 cm, or about 2.9 cm.

The vertical diameter of the second marking surface can be as small as 0.5 cm. In embodiments, the vertical diameter is about 1.0 cm, about 1.2 cm, about 1.3 cm, about 1.4 cm, about 1.5 cm, about 1.6 cm, about 1.7 cm, about 1.8 cm, or about 1.9 cm.

In one embodiment, the horizontal diameter of the second marking surface is about 2.5 cm and the vertical diameter of the second marking surface is about 1.5 cm.

In embodiments, the marking guide is used to mark area for removal of a precancerous or cancerous lesion and reconstruction of the surrounding tissue.

Although FIGS. 1-7 provide exemplary embodiments with circular, elliptical, or oval marking surfaces 110, 210, 310, 410, 120, 220, 320, 420 the marking surfaces can be provided in various shapes and sizes. At least one marking surface can be substantially circular, substantially oval, or polygonal in shape. In polygonal embodiments, at least one marking surface can comprise shapes that are regular or irregular polygons. Embodiments of the surgical marking guide comprise marking surfaces that are substantially triangular in shape, substantially rectangular in shape, or substantially square. Embodiments can comprise marking surfaces with straight or cured edges.

In one aspect, the shape of the marking guide, at least one marking surface or a combination thereof is customizable. By way of non-limiting examples, the shape can be varied depending on the size of the surgical area for graft resection or graft implant, the graft tissue type, the location of the surgical area, or a combination thereof. The of the marking guide, at least one marking surface can also be shaped according to the subject or patient's needs or preferences, including, but not limited, to the subject's age, the subject's comfort, or the subject's weight or height. There can be a number of alternate or additional considerations in customizing the appropriate size and shape of the surgical marking guide 100 that can be apparent to one having skill in the art.

The embodiments of FIGS. 1-7 provide a surgical marking guide with two marking surfaces. However, alternate embodiments can comprise more than two marking surfaces. Some embodiments can comprise a single marking surface. Embodiments can comprise up to 10 marking surfaces. The surgical marking guide can comprise one, two, three, four, five, six, seven, eight, nine, or ten. In embodiments, the marking guide comprises four marking surfaces. In embodiments with four marking surfaces, each marking surface can be positioned opposite another marking surface such that the surgical marking guide is substantially plus-shaped with a marking surface disposed at the termination point of each end of the plus shape.

In various exemplary embodiments, the marking guide 100, 200, 300, 400 can be reusable. In reusable embodiments, the marking guide 100, 200, 300, 400 can be capable of sterilization or can be sterile when provided or obtained. By way the example, the marking guide 100, 200, 300, 400 can be configured to withstand autoclave sterilization, chemical sterilization, or a combination thereof. In alternate embodiments, the marking guide 100, 200, 300, 400 can be configured for a single use. The marking guide 100, 200, 300, 400 can be disposable.

The marking guide 100, 200, 300, 400 can be comprised of any material currently known by those of skill in the art or later developed that is suitable for use in medical or cosmetic procedures. In embodiments, the marking guide 100 is comprised of a medical-grade material. The marking guide 100, 200, 300, 400 can be comprised of a medical grade polymer, metal, or a combination thereof. In embodiments, the marking guide 100 is comprised of sterilizable plastic. The marking guide 100 can be comprised of a surgical marking guide resin. The marking guide 100, 200, 300, 400 can comprise a biocompatible photopolymer resin. The In certain embodiments, the marking guide 100, 200, 300, 400 is comprised of surgical metal. The marking guide 100, 200, 300, 400 can be comprised of stainless steel, titanium, tantalum, gold, platinum, palladium, or any other metal or combination of metals suitable for surgical use.

Embodiments can be created through the process of sculpting, injection molding, casting, or other known methods. Certain embodiments may be printed using three-dimensional printing technology.

Another aspect of the present invention includes a method of using the surgical marking guide 100, 200, 300, 400 in accordance with any embodiment disclosed within this specification or otherwise apparent from the descriptions herein. In one embodiment, the method includes use of the surgical marking guide 100, 200, 300, 400 in the harvest and implant of a nipple areolar complex.

The method can include obtaining a surgical marking guide 100, 200, 300, 400 as described herein. In one embodiment, the first marking surface 110, 210, 310, 410 of the marking guide 100, 200, 300, 400 is depressed against a surgical area of a patient to denote a region to be harvested as a graft material. The graft material can then be removed from the surrounding tissue by surgical means. The second marking surface 120, 220, 320, 420 can then be depressed upon a second surgical area of the patient to denote a region for inset of the graft material. Following removal of the tissue within inset region, the graft material is implanted within the inset region.

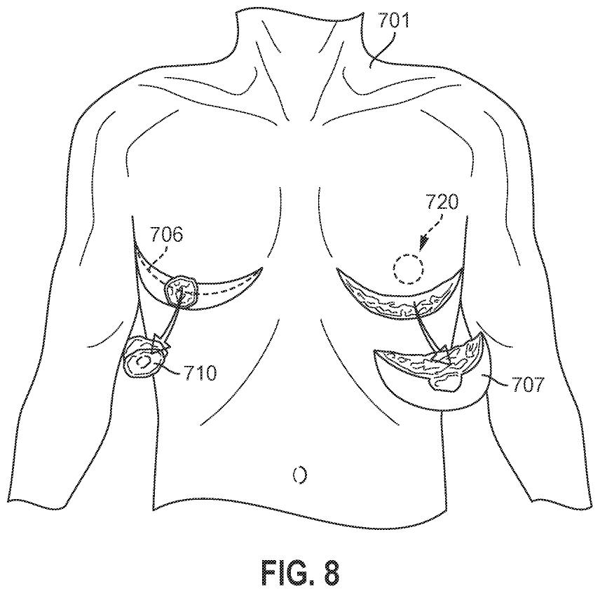

In one embodiment, the graft material comprises a nipple areolar complex and the surgical procedure comprises female-to-male chest reconstruction surgery (see FIG. 8), reconstruction following cisgender gynecomastia, surgical construction of congenitally absent nipples, surgical reconstruction of absent nipples, or a combination thereof.

FIG. 8 provides a view of a schematic view of an exemplary female to male transgender chest reconstruction surgery. As can be seen, the surgical procedure can comprise the removal of a nipple areolar complex graft 710 from a patient 701. The procedure can further comprise an incision 706 along an inframammary fold and subsequent removal of the patient's breast tissue and any excess skin 707. Following removal, the incision 706 can be closed and the new site for the nipple areolar complex graft implantation 720 can be marked. After marking of the implant location, the nipple areolar complex graft 710 can be implanted in the appropriate location 720. As discussed above, the presently disclosed surgical marking guide 100, 200, 300, 400 can be utilized for marking the nipple areolar complex graft 710 for resection, marking of the implant location and orientation 720; confirmation of correct location or orientation of the nipple areolar complex graft 710, or a combination thereof.

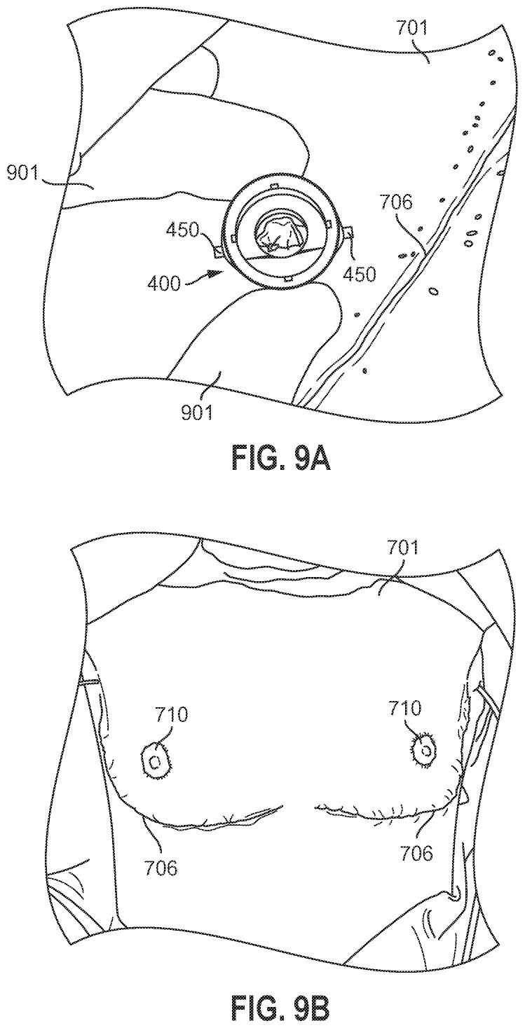

FIG. 9A provides a top view of a surgical marking guide 400 under one embodiment being used to mark an area for implantation of a nipple areolar complex graft material. A practitioner's fingers 901 are shown gripping the surgical marking guide 400 and holding the guide in place over the site for implantation of the graft. The surgical incision 706 made to permit removal of breast tissue and excess skin can be seen extending across the surgical field of the patient 701.

As shown in FIG. 9A, in operation, the orienting extensions 450 are configured to assist the practitioner in properly orienting the surgical marking guide 400. In such embodiments, the practitioner can apply orienting marks along the exterior border of the surgical graft site, wherein each mark corresponds to an orientation extension 450 on the surgical marking guide 400. The practitioner can then place the surgical marking guide 400 on the implantation site, aligning the orientation extensions 450 with the orientation marks to ensure accurate placement of the surgical marking guide 400 and proper inset of the graft material.

FIG. 9B provides a view of a surgical filed of a patient 701 immediately following use of a surgical marking guide as shown in FIG. 9A for bilateral resection and implant of a nipple areolar complex graft material 701 during a female to male transgender chest surgery.

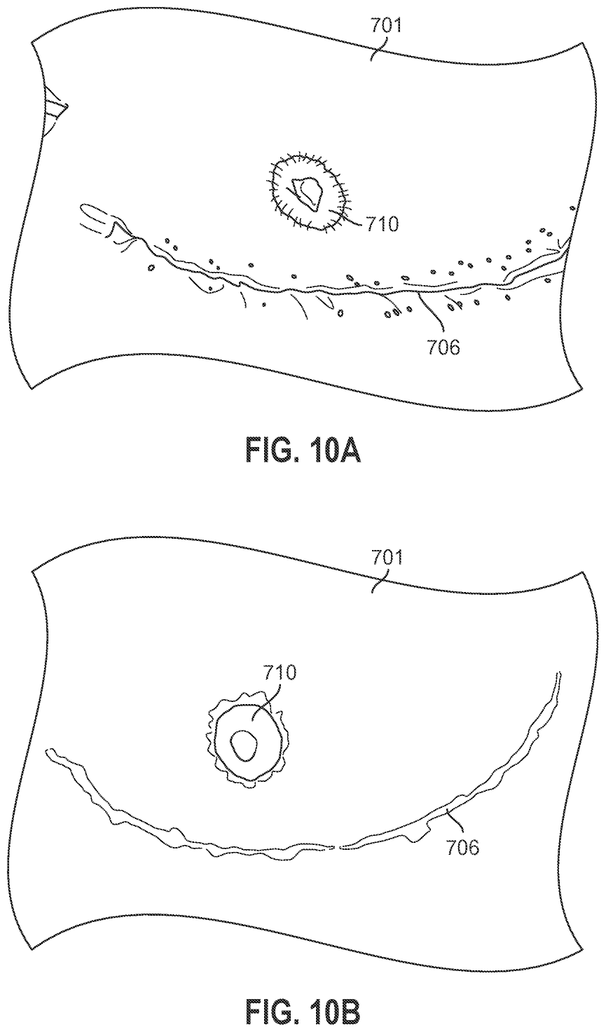

FIG. 10A shows a close-up view of a patient 701 immediately following use of a surgical marking guide under one embodiment for resection and implant of a nipple areolar complex graft material 710 during a female to male transgender chest surgery. The recently closed surgical incision site 706 is shown extending horizontally across the surgical field, below the implanted nipple areolar graft material 710. As can be seen, the newly implanted nipple areolar complex graft 710 can comprise a generally oval shape immediately following implantation.

FIG. 10B shows a close-up view if the patient 701 from FIG. 10A, but three months following the use of the presently disclosed surgical marking guide during a female to male transgender chest surgery. The surgical incision site 706 has healed significantly and the implanted nipple areolar complex graft 710 has developed the prototypical circular shape that is consistent with that desired during such a chest reconstruction surgery.

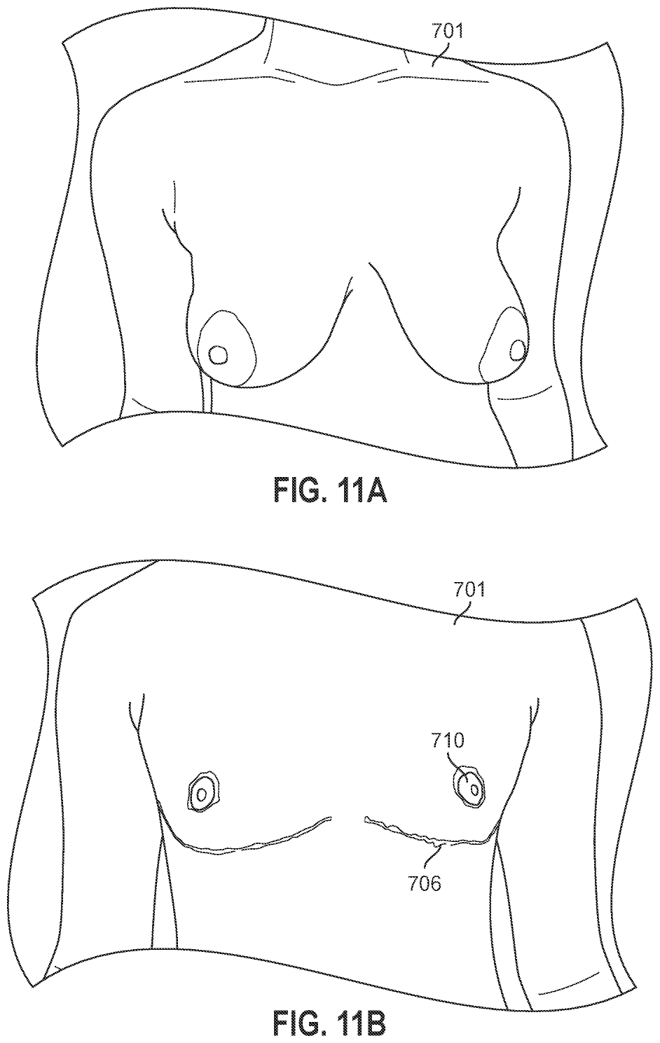

FIG. 11A provides a view of the chest of a patient 701 prior to a female to male transgender surgery under one embodiment. FIG. 11B shows a view of the FIG. 11A patient's chest three months following the use of one embodiment of the presently disclosed surgical marking guide to perform a female to male transgender chest surgery.

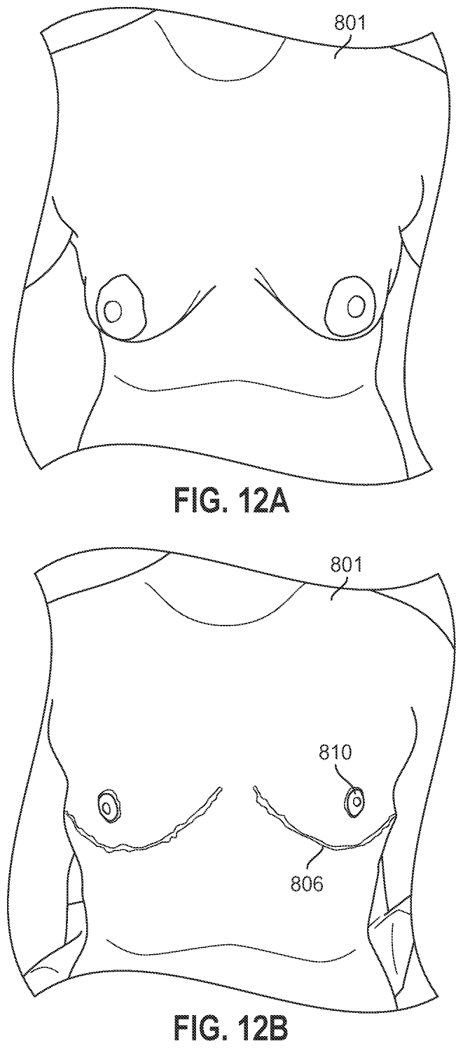

FIG. 12A provides a view of the chest of another patient 801 prior to a female to male transgender surgery, and FIG. 12B shows a schematic representation of the patient 801 from FIG. 12A at three months following the surgical procedure.

As can be seen in the FIG. 11 and FIG. 12 embodiments, the nipple areolar complex graft material 710, 810 assumes a generally circular, masculine shape and the patient's chest has an improved masculine appearance within at least three months following use of the presently disclosed surgical marking guide during a female to male transgender chest reconstruction surgery.

Alternate methods include marking a first surgical area comprising a cancerous or precancerous lesion for removal with the second marking surface followed by removal of the surgical area comprising the cancerous or precancerous lesion. The first marking surface can then be used to mark a second surgical area, wherein the second surgical area comprises a graft material. The graft material can then be removed and implanted within the first surgical area to replace the cancerous or precancerous tissue that was removed.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. A surgical marking guide comprising:
a first marking surface;
a second marking surface; and
a body integral with the first and second marking surfaces and extending therebetween,
wherein a through-opening extends longitudinally from the first marking surface, through the body, and through the second marking surface.

2. The guide of claim 1, wherein
the first marking surface is configured to mark an area for harvest of a nipple areolar complex graft; and
the second marking surface is configured to mark an area for implant of a nipple areolar complex graft.

3. The guide of claim 1, wherein the first marking surface comprises a substantially circular diameter.

4. The guide of claim [0070], wherein the first marking surface comprises a diameter that is about the same as that of an average adult male human nipple areolar complex.

5. The guide of claim [0070], wherein the first marking surface comprises a diameter between about 1 cm and about 4 cm.

6. The guide of claim 5, wherein the first marking surface comprises a diameter of about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, or about 2.9 cm.

7. The guide of claim 1, wherein the second marking surface comprises a substantially oval diameter.

8. The guide of claim 7, wherein the second marking surface comprises a horizontal diameter and a vertical diameter and the horizontal diameter is larger than the vertical diameter.

9. The guide of claim 8, wherein the horizontal diameter is up to 140% of the diameter of an average adult male human nipple areolar complex and the vertical diameter is at least 60% of the diameter of the average adult male human nipple areolar complex.

10. The guide of claim 9, wherein the horizontal diameter is about 114% of the diameter of the average adult male human nipple areolar complex and the vertical diameter is about 68% of the diameter of the average adult male human nipple areolar complex.

11. The guide of claim 8, wherein the horizontal diameter is up to 4 cm and the vertical diameter is as small as 0.5 cm.

12. The guide of claim 8, wherein the horizontal diameter is about 2.0 cm, about 2.1 cm, about 2.2 cm, about 2.3 cm, about 2.4 cm, about 2.5 cm, about 2.6 cm, about 2.7 cm, about 2.8 cm, or about 2.9 cm.

13. The guide of claim 8, wherein the vertical diameter is about 1.0 cm, about 1.2 cm, about 1.3 cm, about 1.4 cm, about 1.5 cm, about 1.6 cm, about 1.7 cm, about 1.8 cm, or about 1.9 cm.

14. The guide of claim 8, wherein the horizontal diameter is about 2.5 cm and the vertical diameter is about 1.5 cm.

15. The guide of claim 1, wherein the first marking surface and the second marking surface each comprise a means for marking the skin of a patient.

16. The guide of claim 15, wherein the means for marking the skin of the patient includes a ridged surface, an inked surface, or a combination thereof.

17. The guide of claim 1, wherein the guide comprises a sterilizable material.

18. The guide of claim 17, wherein the guide comprises a surgical metal, a medical-grade polymer, or a combination thereof.

19. The guide of claim 18, wherein the surgical metal comprises stainless steel, titanium, tantalum, gold, platinum, palladium, or a combination thereof.

20. The guide of claim 1, wherein the guide is reusable.

21. The guide of claim 1, wherein the guide is disposable.

22. A method of grafting a nipple areolar complex to the chest of a patient in need thereof, the method comprising:
marking an area for a nipple areola complex graft using the first marking surface of the surgical marking guide of any one of claims 1-21;
harvesting the nipple areolar complex graft;
marking an area for implant of the nipple areolar complex graft on the patient, wherein marking the area for implant is performed using the second marking surface of any one of claims 1-21;
implanting the nipple areolar complex to the chest of the patient within the marked area for implant.
